(19) United States
(12) Patent Application Publication
(10) Pub. No.: US 2005/0228505 A1
(43) Pub. Date: Oct. 13, 2005

(54) Device and method for treating gastroesophageal reflux disease

(76) Inventors: Douglas A. Cornet, Omaha, NE (US); Aureo DePaula, Goiania (BR); Charles J. Filipi, Omaha, NE (US)

Correspondence Address: HOFFMAN WARNICK & D'ALESSANDRO, LLC, 75 STATE STREET, 14TH FL, ALBANY, NY, 12207, US

(21) Appl. No.: 11/092,190

(22) Filed: Mar. 29, 2005

Related U.S. Application Data
(60) Provisional application No. 60/557,433, filed on Mar. 29, 2004.

(51) Int. Cl. A61F 2/04

(52) U.S. Cl. 623/23.68

(57) ABSTRACT

The present invention relates to a prosthetic anti-reflux valve for treating gastroesophageal reflux disease (GERD) and methods for its use. The preferred methods described provide for transoral insertion of a prosthetic anti-reflux valve and its fixation within the stomach interior. One advantage of such preferred embodiment of the present invention over prior devices is the elimination of the need for fixation of a prosthetic device within the esophagus, thus permitting a more natural and less obstructed, and even unobstructed, function of the esophagus. A first aspect of the invention provides a prosthetic valve for implantation in the stomach of an animal proximate to the esophagus to inhibit reflux of matter from the stomach to the esophagus without substantially inhibiting movement of matter from the esophagus to the stomach comprising a device having an esophageal surface and a gastric surface, wherein the gastric surface is contoured such that the device is thinner around the perimeter than away from the perimeter.

### CROSS REFERENCE TO RELATED APPLICATIONS

The current application claims the benefit of co-pending U.S. Provisional Application No. 60/557,433 filed Mar. 29, 2004, which is hereby incorporated herein by reference.

### BACKGROUND OF THE INVENTION

(1) Technical Field

This invention relates to a device and method for treating gastroesophageal reflux disease and more specifically to a prosthetic anti-reflux device and its non-surgical fixation within the stomach interior.

(2) Related Art

Gastroesophageal reflux is defined by the retrograde flow of stomach acids into the esophagus. Individuals experiencing such reflux on a regular basis (two or more times per week) are often diagnosed with gastroesophageal reflux disease (GERD), which affects approximately 30% of the adult population of the United States.

If left untreated, GERD may cause heartburn, abdominal pain, regurgitation of gastric contents, Barrett's Esophagus, and esophageal cancer. Current treatment methods include antacid medications and surgery.

Antacid medications treat the symptoms of GERD rather than the cause of the disease itself, and may not, therefore, prevent stomach acids from entering the esophagus. Surgical treatments are capable of treating and curing GERD itself, and are generally of two types, either open surgery through the abdomen or chest or laproscopic surgery through the abdominal wall.

Often, surgical treatment involves the implantation of a prosthetic anti-reflux valve within the esophagus in the area of the lower esophageal sphincter (LES). Such prostheses prevent the retrograde flow of stomach acids into the esophagus while permitting the normal flow of liquids and food into the stomach. Examples of such prostheses include U.S. Pat. No. 4,846,836 to Reich and U.S. Pat. No. 5,314,473 to Godin. Other prostheses, such as that described by Dua et al. in U.S. patent application Publication No. 2003/0060894 A1, are further designed to permit retrograde flow in response to the need of the patient to belch or vomit.

In addition, Taylor (U.S. Pat. Nos. 6,544,291 and 6,558,429) has described similar prostheses designed for peroral installation, eliminating the need for open or laproscopic surgical implantation.

Intraesophageal fixation of devices such as those mentioned above, has the potential to interfere with the normal function of the esophagus. In addition, due to the potential for structural failure of such devices, such methods create a significant possibility of morbidity or mortality.

There is, therefore, a continuing need for a prosthetic anti-reflux valve having at least one, and preferably most or all, of the following features: (1) is capable of fixation outside the esophagus, (2) can be installed without the need for open or laproscopic surgery, (3) prevents retrograde flow of stomach acids into the esophagus, (4) permits normal flow of liquids and food through the esophagus and into the stomach, (5) permits retrograde flow in response to high gastric pressure, as where the patient needs to belch or vomit, and (6) minimizes the potential for structural failure of the valve and the attendant consequences of such failure.

### SUMMARY OF THE INVENTION

The present invention relates to a prosthetic anti-reflux valve for treating gastroesophageal reflux disease (GERD) and methods for its use. The preferred methods described provide for transoral insertion of a prosthetic anti-reflux valve and its fixation within the stomach interior. One advantage of such preferred embodiment of the present invention over prior devices is the elimination of the need for fixation of a prosthetic device within the esophagus, thus permitting a more natural and less obstructed, and even unobstructed, function of the esophagus. A first aspect of the invention provides a prosthetic valve for implantation in the stomach of an animal proximate to the esophagus to inhibit reflux of matter from the stomach to the esophagus without substantially inhibiting movement of matter from the esophagus to the stomach comprising a device having an esophageal surface and a gastric surface, wherein the gastric surface is contoured such that the device is thinner around the perimeter than away from the perimeter.

A second aspect of a preferred embodiment of the invention provides a prosthetic valve for implantation in the stomach of an animal proximate to the esophagus to inhibit reflux of matter from the stomach to the esophagus without substantially inhibiting movement of matter from the esophagus to the stomach comprising a gastric surface adapted to respond to gastric pressure (first pressure), pressing the esophageal surface against the stomach wall and preventing reflux of gastric fluids into the esophagus and an esophageal surface adapted to move away from the stomach wall and into the interior of the stomach in response to a second pressure (greater than the first pressure) resulting from the swallowing of liquids or solids, wherein the valve is of sufficient flexibility that it will pucker in response to a third pressure (greater than the first or second pressures), permitting retrograde flow of stomach contents into the esophagus and return to its initial state following cessation of the third pressure.

A third aspect of a preferred embodiment of the invention provides a method of treating gastroesophageal reflux disease, comprising the steps of (a) providing a prosthetic valve having a gastric surface adapted to respond to gastric pressure (first pressure), pressing the esophageal surface against the stomach wall and preventing reflux of gastric fluids into the esophagus and an esophageal surface adapted to move away from the stomach wall and into the interior of the stomach in response to a second pressure (greater than the first pressure) resulting from the swallowing of liquids or solids, (b) positioning the valve within the interior of the stomach whereby the esophageal surface faces the interior of the stomach wall and covers the lower esophageal sphincter, the gastric surface faces the interior of the stomach, the convex portion of the gastric surface is oriented toward the greater curvature of the stomach and the concave portion of the gastric surface is oriented toward the lesser curvature of the stomach, and (c) securing the valve to the wall of the stomach.

The prosthesis of the present invention is composed of a biologically safe, preferably inert material or combination of materials. Such materials may have mechanical properties similar to silicone and, in a particularly preferable embodiment, they are biodegradable.

In one preferred embodiment, the shape of the prosthesis is designed to fit over the opening through which the esophagus empties into the stomach, i.e., the lower esophageal sphincter (LES), which shape is preferably substantially circular (i.e., circular or elliptical), with two distinct surfaces. The first surface is designed to cover the esophageal opening and therefore can be any shape that will accomplish this function. The second surface is substantially convex at one end, gradually becoming concave toward its middle, and finally becoming substantially flat, i.e. the first and second surfaces become parallel or nearly parallel, approaching the opposite end. The concave portion of the second surface permits the prosthesis to respond to changes in pressure within the stomach interior, as described below. Any shape permitting such response to changes in pressure is within the scope of the present invention. This surface is preferably smooth but can be dimpled, rippled, or bumpy.

In this preferred embodiment, the prosthesis of the present invention is designed for fixation at its convex end to the greater curvature of the stomach, wherein the unfixed, flat end of the prosthesis is left free to alternately cover and expose the distal end of the esophagus where it empties into the stomach. The means for attaching the device to the inside lining of the stomach may be any of a number of means used to attach prosthetic devices to stomach, esophageal, or intestinal linings, including, e.g., staples, sutures, and clips. The device is attached only at a portion of the perimeter of the device. To facilitate attachment of the valve to the stomach wall, a thickened portion of the first surface extends beyond the convex end of the second surface, providing a protrusion for attachment to the greater curvature of the stomach.

Under normal gastric pressure (first pressure), the concave portion of the second surface of the prosthesis responds to such pressure such that the unfixed portions of the perimeter of the prosthesis are held against the lesser curvature of the stomach and the body of the prosthesis covers the distal end of the esophagus (first position). In response to orthograde pressures created by normal swallowing of ingested materials (second pressure), the unfixed end of the prosthesis is forced away from the lesser curvature of the stomach (second position), permitting passage of the ingested materials into the stomach. Upon cessation of this second pressure, the unfixed end of the prosthesis returns to its first position, with the unfixed end held against the lesser curvature of the stomach. In response to the high retrograde pressures resulting, for example, from a patient's need to belch or vomit (third pressure), the prosthesis is designed to pucker at its unfixed end (third position), permitting retrograde flow of gasses or stomach contents into the esophagus. Upon cessation of this third pressure, the prosthesis spontaneously returns to its first position, wherein its unfixed end is held against the lesser curvature of the stomach and the body of the prosthesis covers the distal end of the esophagus.

The foregoing and other features of the invention will be apparent from the following more particular description of illustrative embodiments of the invention.

### BRIEF DESCRIPTION OF THE DRAWINGS

The embodiments of this invention will be described in detail, with reference to the following figures, wherein like designations denote like elements, and wherein:

FIG. 1 is a schematic view of a prosthetic anti-reflux valve in accordance with the present invention, positioned within the stomach interior.

FIG. 2 is a side elevational view of the valve of FIG. 1.

FIG. 3 is a side view of the valve of FIG. 1.

FIG. 4 is a planar view of the valve of FIG. 1.

FIGS. 5a through 5c are side elevational views of the valve in response to the absence of gastric pressure, to normal gastric pressure, and to high gastric pressure, respectively.

FIGS. 6a and 6b are schematic and side elevational views of the passage of an endoscope through the valve.

FIGS. 7a through 7c are side elevational and planar views of the preferred embodiment of the present invention.

### DETAILED DESCRIPTION OF THE INVENTION

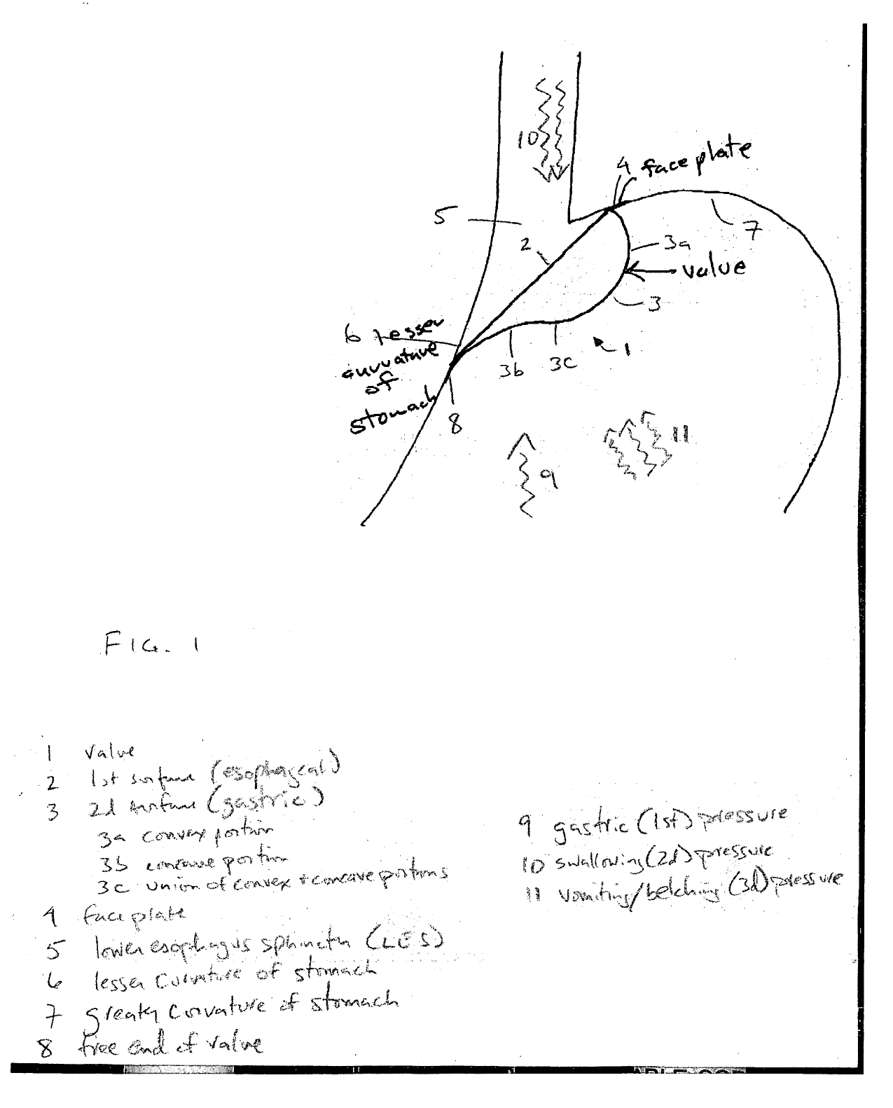

Referring to FIG. 1, a prosthetic anti-reflux valve 1 in accordance with an embodiment of the present invention is illustrated in contact with the greater curvature of the stomach 7 and the lesser curvature of the stomach 6 and in proximity to the lower esophageal sphincter (LES) 5.

The valve 1 is secured to the greater curvature of the stomach 7 at its face plate 4, a thickened portion of the esophageal (first) surface 2 extending beyond the convex end 3a of the gastric (second) surface 3 of the valve. The free end of the valve 8 is unattached and rests against the lesser curvature of the stomach 6 in response to normal gastric pressure 9. In this position, the esophageal (first) surface 2 faces the LES 5 and the gastric (second) surface 3 faces the interior of the stomach.

Referring to FIG. 2, in this embodiment, the anti-reflux valve 1 is generally comprised of an esophageal (first) surface 2, a gastric (second) surface 3, and a face plate 4. The gastric surface 3 is comprised of a convex portion 3a beginning at the junction of the gastric surface 3 and the face plate 4 and extending approximately one third the length of the valve 1 and a concave portion 3b beginning at the termination of the convex portion 3c and extending approximately two-thirds the length of the valve 1, terminating at the free end of the valve 8.

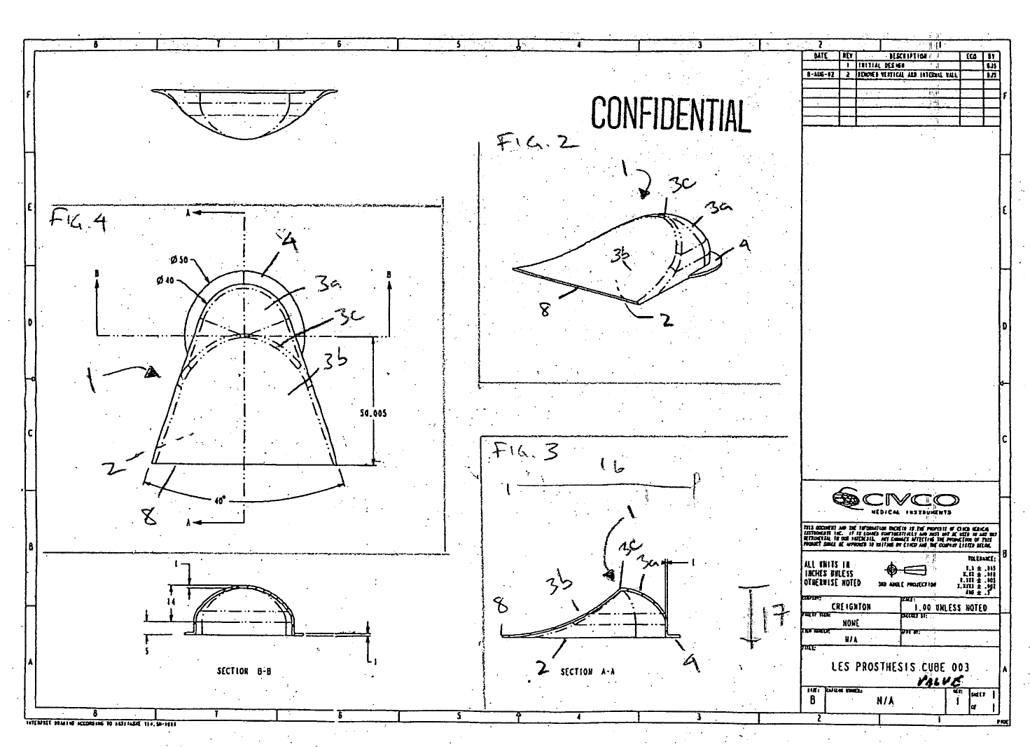

Referring to FIG. 3, in this embodiment, the face plate 4 has a thickness of about 1 mm, preferably 1.5 mm. The valve 1 is thickest at the termination of the convex portion 3c, having an overall thickness 17 of about 20 mm. The thickness of the concave portion 3b gradually decreases from about 20 mm at the termination of the convex portion 3c to about 1 mm at the free end of the valve 8. The overall length of the valve 16 is about 75 mm.

Referring to FIG. 4, in this embodiment, the face plate 4 extends around the convex portion 3a of the valve, following an arc of about 280 degrees. The entire surface of the face plate 4 may be used to attach the valve 1 to the lining of the stomach. The actual portion used may vary depending on the means of attachment.

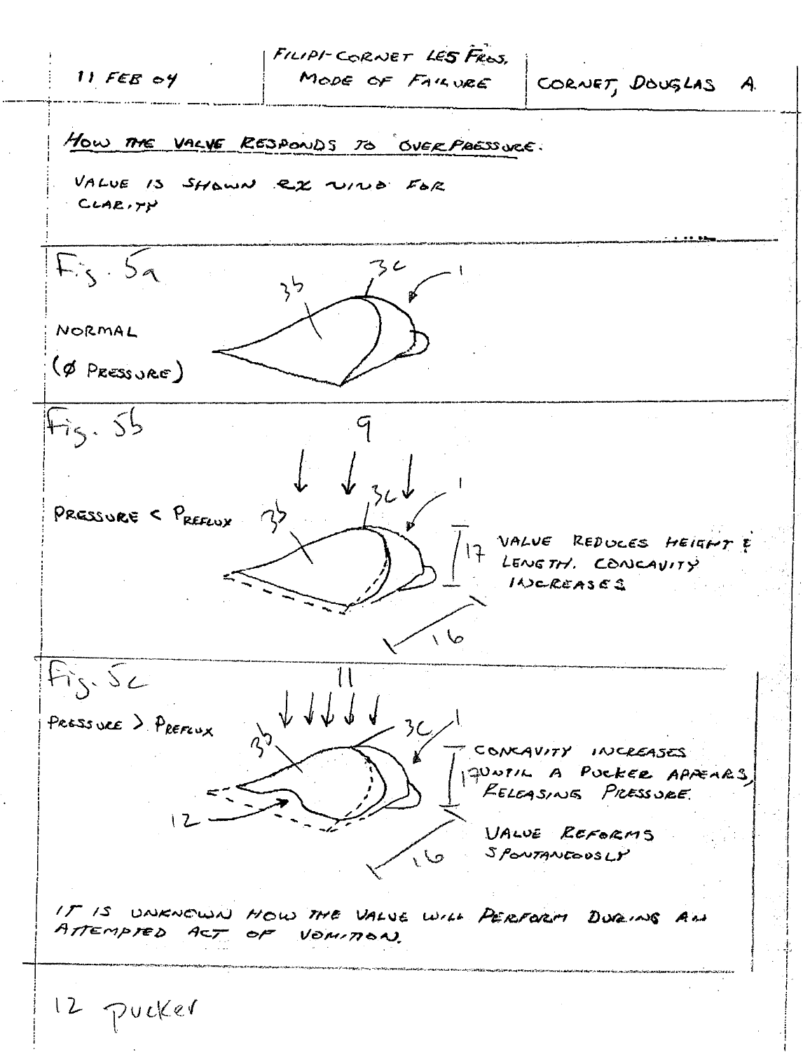

Referring to FIGS. 5a, 5b, and 5c, the shape of a preferred embodiment of the valve 1, and particularly that of its concave portion 3b, will change in response to changes in gastric pressure. The valve in FIG. 5a has its normal shape, i.e. its shape in response to no gastric pressure.

In FIG. 5b, the valve 1 has become deformed in response to normal gastric pressure 9. The concavity of the concave portion 3b has increased and the overall thickness of the valve 17, measured at the termination of the convex portion 3c, has decreased. The increased concavity of the concave portion 3b, results in a decrease in the overall length of the valve 16.

In FIG. 5c, the valve has become more greatly deformed in response to high gastric pressure 11. The concavity of the concave portion 3b has further increased and the overall thickness of the valve 17 has further decreased. Due to the greater concavity of the concave portion 3b, the overall length of the valve 16 has further decreased. In addition, a portion of the free end of the valve 8 has become raised, forming a pucker 12, whereby gastric contents are permitted to flow into the esophagus, reducing the high gastric pressure 11. Upon sufficient reduction of the high gastric pressure 11, the valve 1 spontaneously returns to its position in FIG. 5b.

In a particularly preferred embodiment, the valve 1 would take on the shape in FIG. 5b in response to gastric pressures of about 100 mmHg, resulting in an overall thickness 17 of between 18-20 mm and an overall length 16 of between 70-75 mm. Also in its preferred embodiment, the valve 1 would take on the shape in FIG. 5c in response to gastric pressures above 150 mmHg, preferably before reaching a pressure of 190 mmHg, resulting in an overall thickness 17 of between 18-20 mm and an overall length 16 of between 70-75 mm.

It is possible that in response to very high gastric pressure, a portion of the free end of the valve 8 will prolapse into the LES 5. In such a situation, should the valve not spontaneously return to its position in FIG. 5b following cessation of the very high gastric pressure, the valve can be returned to its position and shape in FIG. 5b by the patient's ingestion of solid or liquid food.

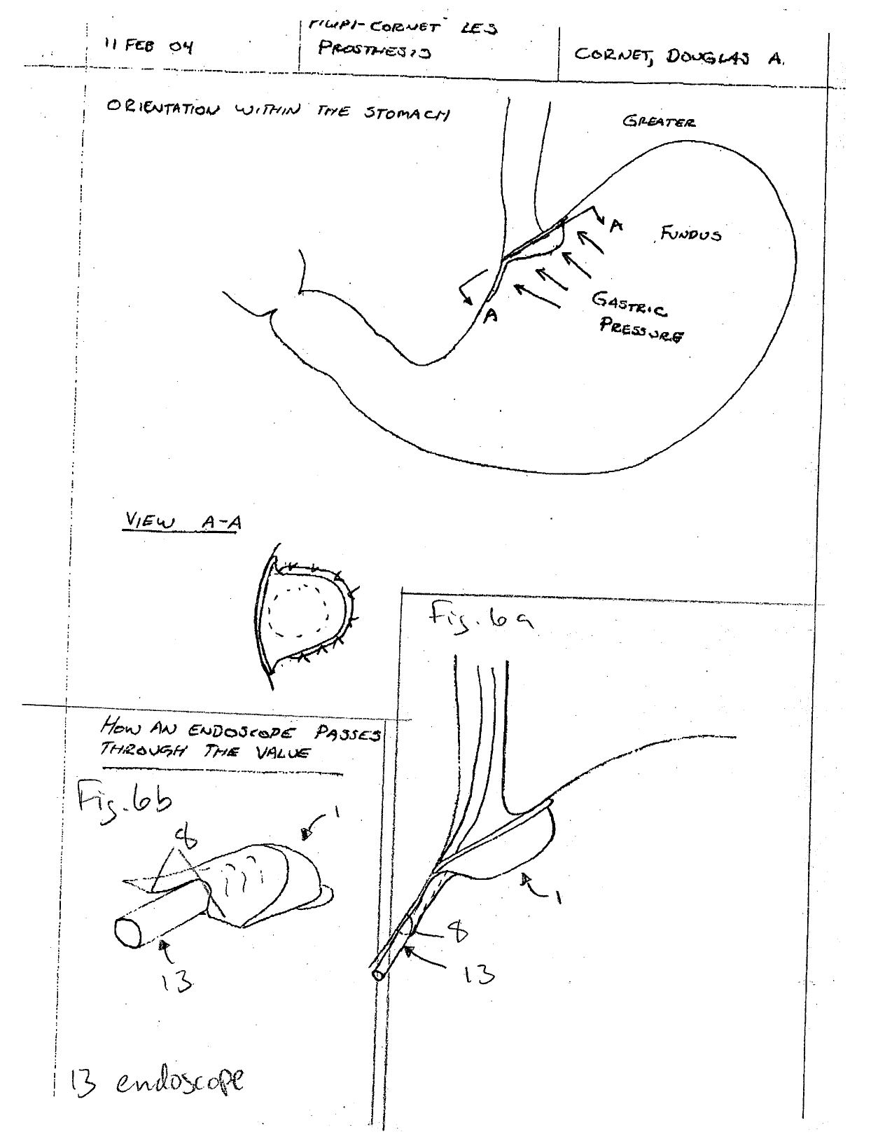

Referring to FIGS. 6a and 6b, one important aspect of the preferred embodiment of the invention is that fixation of the valve 1 within the interior of the stomach will not interfere with the ability of a patient to undergo an endoscopy. An endoscope 13 may be passed between the lesser curvature of the stomach 6 and the free end of the valve 8, permitting passage of the endoscope 13 into the interior of the stomach.

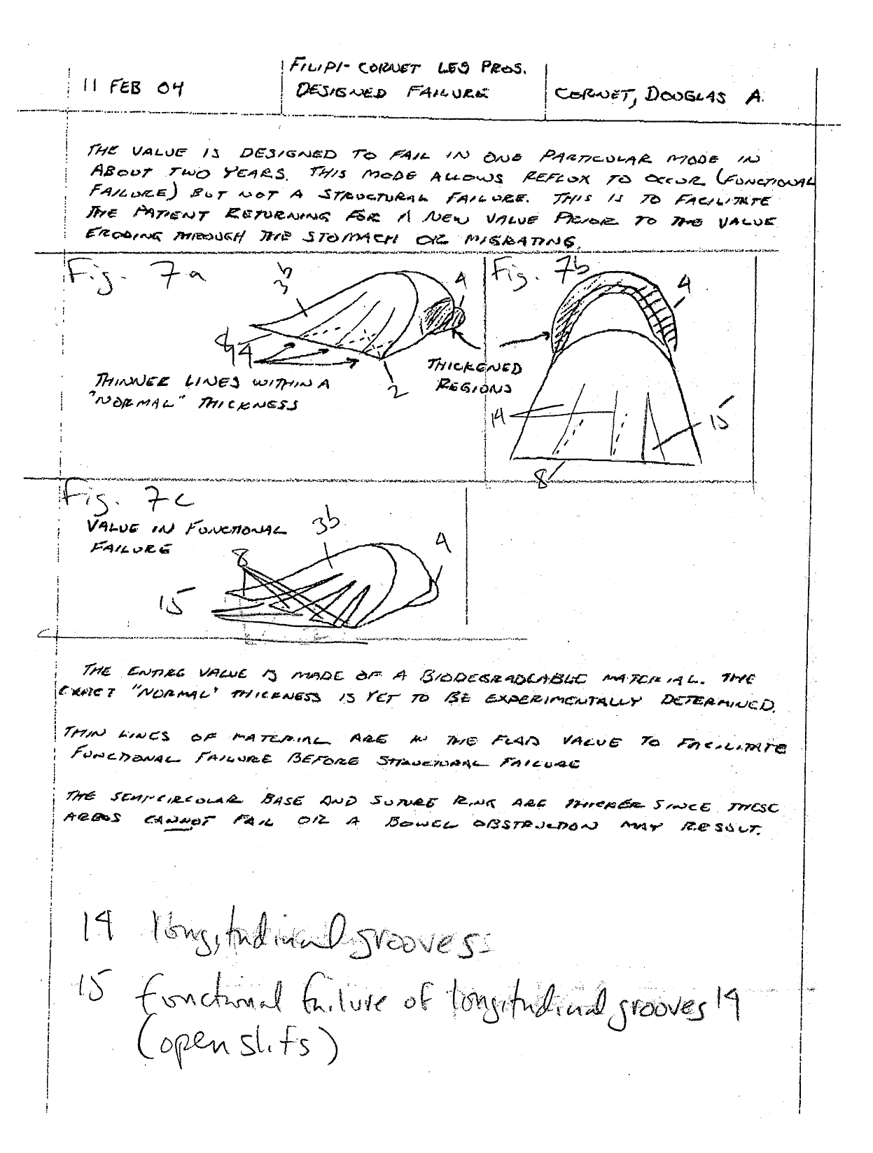

Referring to FIGS. 7a, 7b, and 7c, in one preferred embodiment of the present invention, both the concave portion 3b of the gastric surface and the portion of the esophageal surface 2 adjacent the free end of the valve 8 contain a plurality of longitudinal grooves 14 where the material of the valve is substantially thinner than in other areas of the valve. The longitudinal grooves 14 will be eroded by the gastric contents of the stomach sooner than other areas of the valve. Upon erosion, the grooves form open slits 15, permitting gastric contents to pass through the valve and into the esophagus, resulting in the deliberate functional failure of the valve. Such deliberate functional failure is intended to cause a recurrence of GERD symptoms in the patient, prompting the patient to return to the physician for replacement of the anti-reflux valve. Such functional failure is intended to occur between 18-26 months, preferably between 22-26 months.

The deliberate functional failure and replacement of the valve is intended to supercede any structural failure of the valve, generally typified by erosion of the valve through the stomach lining or detachment of part or all of the valve body from the stomach lining. The consequences of the structural failure of any anti-reflux device, whether that of the current invention or of the intraesophageal devices known in the art, can be severe, including bowel obstruction and death. It is a goal of the preferred embodiment of the present invention, therefore, that any structural failure of the valve be preceded by its functional failure, a recurrence of GERD symptoms in the patient, and replacement of the valve.

Materials suitable for construction of the claimed invention include those typically used for the construction of internal prosthetic devices, such as those disclosed in U.S. patent application Publication 2003/0060894 A1 to Dua et al. and U.S. Pat. No. 6,302,917 to Dua et al., and U.S. Pat. No. 5,861,036 to Godin. Such materials include, for example, polyurethanes, silicones, polyamides, butyl rubbers, nylons, and other urethanes or biocompatible materials. In a preferred embodiment of the claimed invention, the material is biodegradable, with a tensile strength greater than silicone and an elongation similar to silicone.

In a particularly preferred embodiment, the material is polycaprolactone (PCL) having a molecular weight of approximately 50,000 daltons. PCL is non-toxic and tissue-compatible and degrades in vivo in approximately 24 months. This period may be shortened, if necessary, by copolymerizing PCL with one or more of, for example, caprolactone and valeroacetone. In addition, PCL may be elongated 300-500%, permitting movement with tissues to which the valve is attached.

In another particularly preferred embodiment, various valve components are composed of different materials, enabling functional failure of particular valve components at varying times. For example, a number of suitable biodegradable polymers are available from Absorbable Polymers International of Pelham, Ala.. Such polymers may be used, for example, in the formation of a valve body intended to degrade and functionally fail within 18-24 months of implantation and prior to any structural failure of the face plate.

While this invention has been described in conjunction with the specific embodiments outlined above, it is evident that many alternatives, modifications and variations will be apparent to those skilled in the art. Accordingly, the embodiments of the invention as set forth above are intended to be illustrative, not limiting. Various changes may be made without departing from the spirit and scope of the invention as defined in the following claims.

1. A prosthetic valve for implantation in the stomach of an animal proximate to the esophagus to inhibit reflux of matter from the stomach to the esophagus without substantially inhibiting movement of matter from the esophagus to the stomach comprising a device having an esophageal surface and a gastric surface, wherein the gastric surface is contoured such that the device is thinner around the perimeter than away from the perimeter.

2. The prosthetic valve of claim 1, wherein the valve may be positioned such that the esophageal surface faces the esophagus and the gastric surface faces the stomach interior.

3. The prosthetic valve of claim 1, wherein a first portion of the device is inflexible in the direction of the esophageal surface and a second portion is flexible in said direction.

4. The prosthetic valve of claim 2, wherein the surface for attaching the device includes a protrusion from a portion of the edge of the device extending from the esophageal surface to beyond the thickness of the device at said portion.

5. The prosthetic valve of claim 2, wherein a first portion of the device is inflexible in the direction of the esophageal surface and a second portion is flexible in said direction.

6. The prosthetic valve of claim 4, wherein a first portion of the device is inflexible in the direction of the esophageal surface and a portion is flexible in said direction.

7. The prosthetic valve of claim 6, wherein:
the inflexible portion is proximate to the protrusion;
the gastric surface of the inflexible portion is convex relative to the esophageal surface;
the flexible portion is distal to the protrusion; and
the gastric surface of the flexible portion is concave relative to the esophageal surface.

8. The prosthetic valve of claim 7 wherein:
a first end of the convex portion of the gastric surface begins at a first end of the valve and extends approximately one-third the total length of the valve;
a first end of the concave portion of the gastric surface begins at a second end of the valve opposite the first end of the valve and extends approximately two-thirds the total length of the valve; and
a second end of the concave portion of the gastric surface adjoins a second end of the convex portion of the second surface.

9. The prosthetic valve of claim 1, wherein the valve includes at least one of the following: a polyurethane, a silicone, a polyamide, a butyl rubber, a nylon, a urethane, a polycaprolactone, a polycaprolactone-caprolactone copolymer, and a polycaprolactone-valeroacetone copolymer.

10. The prosthetic valve of claim 1, wherein at least one of the gastric surface and the esophageal surface includes an area of material thinner than that of other areas of the valve, whereby said area of thinner material will erode sooner than said other areas of the valve.

11. The prosthetic valve of claim 10, wherein the area of thinner material is about 1.0 mm thick and the other areas of the valve are between about 1.0 and about 1.5 mm thick.

12. A prosthetic valve for implantation in the stomach of an animal proximate to the esophagus to inhibit reflux of matter from the stomach to the esophagus without substantially inhibiting movement of matter from the esophagus to the stomach comprising:
a gastric surface adapted to respond to gastric pressure (first pressure), pressing the esophageal surface against the stomach wall and preventing reflux of gastric fluids into the esophagus; and
an esophageal surface adapted to move away from the stomach wall and into the interior of the stomach in response to a second pressure (greater than the first pressure) resulting from the swallowing of liquids or solids,
wherein the valve is of sufficient flexibility that it will pucker in response to a third pressure (greater than the first or second pressures), permitting retrograde flow of stomach contents into the esophagus and return to its initial state following cessation of the third pressure.

13. The prosthetic valve of claim 12, wherein:
the first pressure is between 0 and 100 mmHg;
the second pressure is between 100 and 150 mmHg; and
the third pressure is greater than or equal to 150 mmHg.

14. The prosthetic valve of claim 12, wherein the valve includes at least one of the following: a polyurethane, a silicone, a polyamide, a butyl rubber, a nylon, a urethane, a polycaprolactone, a polycaprolactone-caprolactone copolymer, and a polycaprolactone-valeroacetone copolymer.

15. The prosthetic valve of claim 12, wherein at least one of the gastric surface and the esophageal surface contains an area of material thinner than that of other areas of the valve, whereby said area of thinner material will erode sooner than said other areas of the valve.

16. The prosthetic valve of claim 15, wherein the area of thinner material is about 1.0 mm thick and the other areas of the valve are between about 1.0 and about 1.5 mm thick.

17. A method of treating gastroesophageal reflux disease, comprising the steps of:
(a) providing a prosthetic valve having:
a gastric surface adapted to respond to gastric pressure (first pressure), pressing the esophageal surface against the stomach wall and preventing reflux of gastric fluids into the esophagus; and
an esophageal surface adapted to move away from the stomach wall and into the interior of the stomach in response to a second pressure (greater than the first pressure) resulting from the swallowing of liquids or solids;
(b) positioning the valve within the interior of the stomach whereby the esophageal surface faces the interior of the stomach wall and covers the lower esophageal sphincter, the gastric surface faces the interior of the stomach, the convex portion of the gastric surface is oriented toward the greater curvature of the stomach and the concave portion of the gastric surface is oriented toward the lesser curvature of the stomach; and
(c) securing the valve to the wall of the stomach.

18. The method of claim 17, wherein the valve is of sufficient flexibility that it will pucker in response to a third pressure (greater than the first or second pressures), permitting retrograde flow of stomach contents into the esophagus and return to its initial state following cessation of the third pressure.

19. The method of claim 17, wherein the valve includes at least one of the following: a polyurethane, a silicone, a polyamide, a butyl rubber, a nylon, a urethane, a polycaprolactone, a polycaprolactone-caprolactone copolymer, and a polycaprolactone-valeroacetone copolymer.

20. The method of claim 17, wherein the valve is adapted to functionally fail prior to its structural failure.
